(19) United States
(12) Patent Application Publication
(10) Pub. No.: US 2009/0005746 A1
(43) Pub. Date: Jan. 1, 2009

(54) Suction System

(76) Inventors: Brian Nielsen, Gorlose (DK); Jesper Mads Bartroff Frederiksen, Vedbaek (DK); Truels Sterm Larsen, Frederiksberg (DK); Erik Lund Jepsen, Vaerlose (DK); Niels Hvid, Vedbaek (DK); Klaus Budig, Olstykke (DK)

Correspondence Address: JACOBSON HOLMAN PLLC, 400 SEVENTH STREET N.W., SUITE 600, WASHINGTON, DC, 20004, US

(21) Appl. No.: 12/087,522

(22) PCT Filed: Feb. 1, 2007
(86) PCT No.: PCT/DK2007/000054

§ 371 Date: Jul. 9, 2008

(30) Foreign Application Priority Data
Feb. 2, 2006 (DK) PA 2006 00147
Feb. 2, 2006 (DK) PA 2006 00148
Feb. 2, 2006 (DK) PA 2006 00149
Jul. 24, 2006 (DK) PA 2006 01010

(51) Int. Cl. A61M 1/00

(52) U.S. Cl. 604/315

(57) ABSTRACT

A suction system for removal of exudate from a wound comprises a wound cover which is attachable to a wound circumference of a living being thereby to form an enclosure, and a pump in fluid communication with the enclosure to provide a pressure difference between a negative pressure in the enclosure and an air pressure of an ambient space. A deflectable member is arranged such with respect to the enclosure that the pressure difference may cause deflection of the deflectable member, and a pressure sensing device is located outside the enclosure and arranged to provide a signal in response to the pressure or the deflection of the deflectable member. The deflectable member forms an essentially air tight barrier between the sensor and the enclosure.

### TECHNICAL FIELD

The present invention relates to a suction system for removal of exudate from a wound and thus for enhancing wound healing. The system comprises a wound cover which is attachable to a wound circumference of a living being to form an enclosure, a pump in fluid communication with the enclosure to provide a pressure difference between a negative pressure in the enclosure and an air pressure of an ambient space, a deflectable member adapted to move or deflect based on the pressure difference, and a pressure sensing device located outside the enclosure and adapted to provide a signal based on either pressure or deflection of the deflectable member.

### BACKGROUND OF THE INVENTION

It has been found that fluid drainage of wounds promotes tissue growth and thereby facilitates a reduced healing time. The treatment has been exercised for many years and various therapeutic apparatus for providing suction to a wound have been developed.

U.S. Pat. No. 6,648,862 describes a vacuum desiccator using a canister which contains a trapping agent, and WO 97/18007 discloses a portable wound treatment apparatus with a canister and a pump arranged in a housing which promotes portable use, e.g. wearable on a harness or via a belt.

In the known apparatuses, a wound cover is fixed in a sealing manner to the skin of a living being so that an enclosure is formed around the wound. The cover is connected to a pump, and suction is applied. The suction forces exudate from the enclosure to a receptacle.

To control operation of the vacuum system it may be desirable to measure a pressure in the system in order to provide a control signal for operation of the pump.

In multi-user pump systems for the treatment of wounds, cross-contamination between users is to be avoided.

### DESCRIPTION OF THE INVENTION

It is an object of preferred embodiments of the invention to improve the existing systems, and in particular to provide an increased safety against cross contamination and infection of the treated living being. It is another object of preferred embodiments of the invention to provide a system, in which a pressure can be detected in a manner, which minimizes the risk of cross contamination between patients and the risk of contamination of durable (i.e. reusable) hardware, such as a pressure sensor.

In a first aspect, the invention provides a suction system for removal of exudate from a wound, the system comprising:
a wound cover which is attachable to a wound circumference of a living being thereby to form an enclosure,
a pump in fluid communication with the enclosure to provide a pressure difference between a negative pressure in the enclosure and an air pressure of an ambient space,
a deflectable member arranged such with respect to the enclosure that the pressure difference may cause deflection of the deflectable member, and
a pressure sensing device located outside the enclosure and arranged to provide a signal in response to the pressure or the deflection of the deflectable member,
   wherein the deflectable member forms an essentially air tight barrier between the sensor and the enclosure.

In a second aspect, the invention provides a method of removing exudate from a wound, said method comprising the steps of:
forming an enclosure around a wound by attaching a wound cover to a circumference of the wound,
providing a pressure difference between a negative pressure in the enclosure and an air pressure of an ambient space,
arranging a deflectable member such with respect to the enclosure that the pressure difference causes movement or deflection of the deflectable member, and
providing a signal in response to movement or deflection of the deflectable member,
   wherein the deflectable member is arranged to form a bacterial barrier between the sensing device and the enclosure, and wherein deflectable member may be part of the sensing device. In particular, the method may comprise the step of controlling the pump based on the pressure signal to provide a desired negative pressure in the wound enclosure.

Accordingly, the invention provides a system wherein the deflectable member separates the pressure-sensing device from exudate in the enclosure.

Since the deflectable member not only deflects and thereby facilitates measuring of the pressure but also separates the pressure-sensing device from the exudate, the risk of contamination of the drive unit is reduced, and the sensing device can be reused numerous times without sterilisation. Accordingly, the costs may be reduced while the safety is increased. Preferably, the deflectable member forms a barrier to bacteria, vira, gas and liquid.

In preferred embodiments of the invention, the pump comprises at least one disposable part including e.g. a container for collecting liquid pumped from the wound, and at least one durable (i.e. reusable) part including e.g. drive components for the pump. In such embodiments, the pressure sensing device is preferably arranged in the durable part, and the deflectable member in the disposable part to thereby reduce both cost and risk of contamination.

In general, the wound cover, the deflectable member, and other components of the system which may become contaminated during the treatment may be disposable which means that the components are designed to be used for a short period of time relative e.g. to a reusable drive unit which actuates the pump. Upon placement of the wound cover, a substantially airtight seal may be formed over the wound site to prevent vacuum leakage. Placing a cover over the wound may provide such a seal, such that the cover adheres to the healthy skin surrounding the wound site, while maintaining an airtight seal over the wound itself. In the present context, “airtight” and “substantially airtight” should be understood to mean that a negative pressure may be maintained at the wound, at least during operation of the pump.

The deflectable member should preferably be essentially airtight and thereby prevent diffusion of bacteria and exudate in general through the membrane. The deflectable member may be made of a material that is predominantly impermeable to air and other gases. As no polymers are completely impermeable to gases over longer periods, the term “predominantly impermeable” as used herein means that permeation during one treatment with the deflecting member is negligible for the measurement. The deflectable member may in particular be essentially impermeable to bacteria. Bacteria typically have a diameter of about 0.2 μm. Hence, the barrier may be essentially impermeable to particles larger than 0.02 μm to provide a safety factor of about 10. The deflectable member may also be impermeable by virus, and thus act as a barrier between the single use environment and the surrounding environment. Though small in size (20-300 nm) virus will not be able to pass through a solid material such as an impermeable deflecting member. One example of a material is nitrile rubber used for laboratory gloves. The deflectable member may be made from a flexible polymer material. Plastic materials such as PE, PP, and PVC may be selected since they are typically inexpensive, and they are suitable for injection moulding or blow moulding. The deflectable member may also be made from a material selected from the group consisting of: thermoplastic elastomers (TPU, SIS, SBS and SEBS), thermosetting or vulcanizing elastomers such as synthetic and natural rubber, latex, glass, metal, and ceramics. In any case, the member should be designed to deflect upon a pressure difference of the above-mentioned range, i.e. 10-600 mmHg or preferably in the range of 10-200 mmHg.

The present considerations regarding embodiments of the deflectable member and the requirements thereto also apply by analogy to embodiments of the pumping element (i.e. pump head), such as a tube for use in a peristaltic pump, a diaphragm for use in a diaphragm pump or a piston for use in a piston pump.

In general, the principle of deflection may be based on
i) elastic expansion of a membrane, or
ii) a change in the shape of the deflectable member, i.e. e.g. by bending of the material, such as by elastic bending, or
iii) movement of one element relative to another element of the deflectable member.

Ad i). If the deflection is based on elastic expansion, the deflectable member may include a relatively thin membrane or diaphragm, e.g. a balloon or a diaphragm which is stretched over a capsule, or which is stretched over an open end of a tube, or which is stretched over two or more spaced discs and thus forming a flexible wall in a cylinder etc., the deflectable member being in fluid communication with the enclosure.

Ad ii). If the deflection is based on bending of the material, the deflectable member may include a bellow shaped member which can expand and contract in one direction based on the pressure difference, or the deflectable member may include a Bourbon tube, i.e. a tube which changes its shape depending on the internal pressure.

Ad iii). If the deflection is based on elements moving relative to each other, the elements may include a “rolling diaphragm” or a piston and cylinder arrangement or a liquid string in a tube.

Irrespective of the principle of deflection, the degree of deflection for a certain pressure difference, i.e. the resistance of the deflectable member against the deflection may be controlled by the properties of the selected materials, the dimensions of the deflectable member or by a separate force providing structure which influences the deflection. As an example of such a structure, a spring, e.g. a helically coiled spring could be located to influence the deflection.

The deflectable member may be utilized in two different ways:
a) as a passive member, which deflects without any noticeable resistance and acts solely as an air tight barrier between the enclosure and the pressure sensing device, or
b) as an active member, the deflection of which is in balance with the pressure in the wound enclosure and which is detected via a sensing device, the deflectable member thus being a part of the pressure sensing device.

Ad a). The deflectable member is connected to a pressure sensor on the side opposite the enclosure via a measuring conduit or chamber. As the deflectable member moves without noticeable resistance or deflects almost stresslessly, it will take up a position to provide the same pressure on both sides of the deflectable member, such that the pressure can accurately be measured with any known kind of pressure sensors through the barrier.

In general, the sensor may include any type of sensor, which is capable of measuring a pressure difference of the kind in question. For example, the sensor may comprise an element selected from the following group:
a strain gauge element,
a piezo-resistive element,
a piezo-electric element,
a Bourbon tube
micro electro mechanic systems (MEMS or solid state MEMS),
a vibration element (silicon resonance),
a variable capacitance element, and
a Micro Pirani vacuum gauge.

Ad b): The pressure-sensing device may be located outside the enclosure and is adapted to provide a signal based on deflection of the deflectable member. In general, the sensing device may be of any kind, which is capable of measuring a dimension, a distance, a deflection, a movement or a force.

The applied sensing principle may be based on contact between the sensing device and the deflectable member, i.e. contact measurement, or it may be independent upon direct contact, i.e. contactless measurement.

The sensing device for contact sensing may comprise an element selected from the following group:
a piezo-resistive element,
a plezo-electric element,
a vibration element (silicon resonance),
a variable capacitance element, and
mechanical measurement of deflection e.g. by use of:
   a strain gauge element,
   a linear motion position sensor,
   a potentiometer,
   a force sensor, or
   a force sensitive resistor element (FSR).

Contactless measurement may be based on:
ultrasonic reflection,
reflected light (IR-LED/laser diode),
triangulation (IR-LED/laser diode and a PSD—Position Sensing Device),
differential variable reluctance transducer (DVRT or LVDT—with a core in a coil).

In any case, the signal is preferably an electrical signal, which can be used to monitor the negative pressure in the enclosure via a reading instrument or a display and/or to control the pump to provide a desired negative pressure.

The cost of a sensor or a sensing device is often relatively high compared to the cost of the disposable components. Further a disposable sensor includes electronic parts, which require power supply and means for transfer of signals to a durable (reusable) display or control unit. Accordingly, it may be an advantage to have the sensing device separate from the deflectable member, and thus to allow the deflectable member to be disposed after each use. Accordingly, the deflectable member is preferably detachably connectable in a leak tight manner to a pressure sensor, or it may be arranged to engage a contact sensing device or to engage in correct position relative to a contactless measuring device, e.g. via a snap connection system which in an easy manner facilitates correct positioning of the deflectable member relative to the sensing device. To ensure correct measurements, the connection system may be arranged to prevent use of the pump unless the position of the deflectable member is correct relative to the sensor.

In one embodiment, the deflectable member forms a wall part of the wound cover, or it forms part of elements, which are in fluid communication with the enclosure, e.g. it forms part of a tube, which extends from the enclosure.

The deflectable member may form part of the wound cover, or the deflectable member may form a wall-part of a pressure signal conduit, which is in fluid communication with the enclosure. The pressure signal conduit allows the sensor to be located remote from the wound, and the sensor may thereby be comprised in a drive unit for the actuation of the pump.

The pressure signal conduit may be formed by or embedded in a medical tube. In one embodiment, the tube forms several lumen, wherein one lumen forms the pressure signal conduit, and another lumen forms the drainage conduit. As an alternative to the use of a multi lumen tube, the pressure signal conduit and the drainage conduit may be formed by individual medical tubes, and the tubes may be joined for enhancing the handling of the system and for enhancing connection of the wound cover, the pump and the pressure sensor. In order further to enhance the connection of the parts, the multi lumen tube or the single lumen tubes may comprise coupling means which, in one coupling action, connect the pump to the drainage conduit and the pressure signal conduit to the sensor. Analogously, the decoupling may be obtained for both tubes by a single decoupling action.

Since the signal conduit, contrary to the drainage conduit, merely conducts a pressure signal and not a flow, liquid substances such as exudate are not disposed to enter into the pressure signal conduit. To increase the reliability of the system, the signal conduit may, however, form an inlet into the enclosure, which inlet comprises a separation structure preventing entrance of liquid substances and exudate into the signal conduit. The separation structure may e.g. be a highly flexible barrier e.g. the above-mentioned deflectable member or a second “stressless” deflectable member.

To provide a fast and precise signal transmission and to limit the deflection or movement of the deflectable member, it is preferred to provide the pressure signal conduit with a relatively small volume, preferably smaller than the volume of the drainage conduit. Accordingly, the signal conduit may preferably have a smaller cross-sectional area than the drainage conduit.

In one embodiment the deflectable member is an essentially stresslessly deflectable part connected to a measuring conduit, and the sensing device is a pressure sensor.

In another embodiment the deflectable member comprises an essentially stresslessly deflectable part and a force-providing spring structure, which may be an integral part of the deflectable member or a separate element, and the sensing device is a contactless distance sensor.

The method according to the second aspect of the invention may further comprise a procedure to calibrate the signals at start up after replacement of disposable deflective member.

In the system according to the invention, the pump may comprise a pump head, and the system may further comprise a separating means comprising a fluid inlet and a gas outlet, the pump head being connected to the gas outlet of the separating means. The pump head may be detachably attached to the drive unit, so that in use the fluid inlet of the separating means is connected to a wound cover, e.g. via a conduit.

The separating means separate fluid pumped from the wound from gas before pumped matter enters the pump. Hence, it may be at least substantially prevented that the fluid and possibly also solid matter pumped from the wound enters the pump, whereby wear on the pump and the risk of occlusion of the pump may be avoided. Moreover, pumps which do not pump liquid and solid matter may be simpler, require less driving force due to less viscous resistance in the pump, and manufactured at a lower cost than pumps, which are to pump liquid and/or solid matter. The pump may comprise a peristaltic pump, in which negative pressure is generated by successive contraction and expansion of a flow cross-section of a tube, contraction and expansion being e.g. achieved by a rotatable element with one or more protruding portions acting on an outer surface of the tube to thereby cause the flow cross-section in the tube to successively contract and expand. Alternatively, the pump may include at least one reciprocatable element, such as a piston or diaphragm, arranged to cause successive contraction and expansion of a pumping cavity, at the inlets and outlets of which there are preferably provided respective one-way valves.

As used herein, the terms “pumping element” and “pump head” designate such elements, through which pumped matter and/or gas flows during pumping. Hence, the terms “pumping element” and “pump head” do, in the present context, not normally include drive elements for driving the pumping element or pump head. The terms “pump” and “means for applying negative pressure” designate any pumping structure or pump of performing a pumping action, including drive elements for driving the pumping element or pump head.

In a preferred embodiment, the means for applying negative pressure comprises two detachably attached main parts, a pump head and a drive unit. The pump head may e.g. include the piston or diaphragm mentioned above, and the drive unit may include those elements required to cause the pump head to reciprocate or cause a pumping action. Preferably, a liquid and/or gas tight seal is provided between the pump head and the drive unit, so that gas and/or liquid is prevented from entering the drive unit. For example, the pump head, including e.g. a piston or a membrane, may seal circumferentially against a wall partition. Contamination of the drive unit may thereby be avoided, or the risk of contamination of the drive unit may be reduced. Hence, only the pump head needs sterilization between uses of the device with different patients. Alternatively, the pump head may be integrated in a disposable unit. As the pump head is detachably attached to the drive unit, the pump head may be easily sterilized and/or exchanged.

The pump may produce a pressure difference, which is sufficient to draw liquids away from a wound, e.g. a negative pressure in the range of 10 to 600 mm Hg relative to the surrounding atmosphere.

The wound cover may be occlusive or semi-occlusive, e.g. being vapour permeable but water impermeable. In one embodiment of the invention the wound cover is semi permeable by providing the wound cover with a semi permeable cover foil. In this relation, the term semi permeable means being aqueous vapor permeable. In one embodiment, the wound cover is kept in place by means of adhesive on a part of the surface or the entire surface of the wound cover. In another embodiment of the invention, the wound cover is kept in place by the negative pressure applied by the system.

Providing the means for applying negative pressure as two detachable parts presents several advantages. The pump head typically comprises mainly mechanical parts, while the drive unit also comprises more sensitive elements such as for example an electric motor, electronic circuitry and possibly a control unit. It is in the pump head that a negative pressure or even vacuum is created. Since the parts are detachable from each other, and since the drive unit is isolated with regards to contact with potentially virus- or bacteria-carrying air or exudates, the drive unit can be reused without any risk of transferring virus or bacteria to a possible next user of the system. Thus, the system according to the invention provides a cost-advantage as a main part of the system, namely the drive unit of the means for applying negative pressure, can be reused. As the drive unit is typically the most expensive part due to its electronic components, this cost-advantage is substantial in relation to the total cost of the system.

The pump head and the drive unit are detachably attached to each other. The connection between the pump head and the drive unit may be provided in several different ways. The skilled person will appreciate suitable ways to provide such a connection.

In a preferred embodiment of the invention, the pump head is disposable. The detachable pump head is disposable and can be replaced, while the drive unit can be reused as it has not been in contact with neither exudates nor potentially virus- or bacteria-carrying air. As the pump head mainly comprises mechanical and simple parts, it is possible to manufacture the pump head from relatively inexpensive materials, which reduces the cost of the pump head.

The pump head may e.g. be integrated in a canister or container for collecting fluid from the wound or treatment site. The container may e.g. Include an inlet port connecting to a conduit providing a flow passage to the treatment site. In addition, the container may provide an outlet connecting to an inlet port of the pump. In the device of the first aspect of the invention, and in the system of the second aspect of the invention, an outlet port of the pump preferably connects to a gas discharge opening, through which pumped gas may be pumped to an ambient atmosphere. In the pump of the third aspect of the invention and in the system of the fourth aspect of the invention, an outlet port of the pump may either connect to a gas discharge opening, in case liquid is separated from gas upstream of the pump, or to a container for collecting liquid, in case liquid is not separated from gas upstream of the pump. In this gas, a gas discharge opening is preferably provided downstream of the pump, e.g. in the collecting container. Herein, the terms “upstream” and “downstream” are used to designate locations with respect to the flow direction. Hence, if a first location or element is said to be upstream of a second location or element, this means that the flow of liquid and/or gas reaches the first location or element before it reaches the second location or element. Analogously, if a first location or element is said to be downstream of a second location or element, this means that the flow of liquid and/or gas reaches the second location or element before it reaches the first location or element.

The collecting container may be connected to or integrated with a drive unit housing driving components for the pump. The collection container is preferably a disposable element, which is releasably attached to the drive unit.

As described above, the container can be provided as an integrated part of the separating means, or it can be provided as a separate unit, which is to be assembled with the separating means. The container may be provided for collecting and containing exudates collected from a wound. The container may be provided in several different sizes so as to accommodate different needs of different users, e.g. small volume containers for increased mobility and comfort and large volume containers for large amounts of exudates and/or to be used by bedridden users. The container may be provided in several forms as will be appreciated by the person skilled in the art. It may be provided as a relatively rigid container in the shape of a box, or it may be provided as a flat container of relatively thin material making the container able to bend or fold in order to allow and follow movement of a mobile user.

In one embodiment of the invention, the separating means comprises an elongate separating part with a fluid inlet, a fluid outlet, and a gas outlet.

In another embodiment of the invention, the separating means is an integrated part of the conduit.

One or more filters are preferably provided in or upstream of the gas discharge opening. The filter or filters may be provided before the pump head, i.e. upstream thereof, and/or at an outlet thereof. For example, an odour (i.e. deodorizing) filter and/or a bacterial filter may be provided. Any other or alternative filter, including odour and/or bacterial filters may be provided, e.g. one or more active coal (carbon) filters. The filter may be hydrophobic and/or lipophobic. In one embodiment the filter is placed before the pump head. The filter may be placed at or close to the gas outlet of the separating means to prevent liquid or solid particles from entering the pump head. In one embodiment the filter is a bacterial filter, which is hydrophobic and preferably also lipophobic. Thus, aqueous and oily liquids will bead on the surface of the filter. During normal use there is sufficient airflow through the filter such that the pressure drop across the filter is not substantial.

In embodiments, in which the pump head (i.e. pumping element) is detachably mounted to the drive components of the pump, the filter(s) may be provided downstream of the pump head, as the pump head may easily be exchanged with a new one or temporarily removed for cleaning or sterilization. Hence, provided that the pump head is sealed towards the drive components of the pump, the pump head may be deliberately contaminated, and even in the event of failure of the filter(s), the drive components of the pump are not at risk.

The container, which may be included in the separating means, may include at least one inlet for fluid and gas to enter the container, and at least one outlet for gas to exit the container into the means for applying negative pressure. In such an embodiment, the inlet and outlet may be arranged such with respect to the container and the pump that liquid is separated from gas upstream of the pump. A gas outlet allowing gas pumped through the pump to escape should be comprised in or connected to the pump.

The container may comprise an absorbent element, e.g. for absorbing liquid pumped from the wound. The absorbent element may e.g. comprise a gelling agent, a desiccant, or so-called super absorbent particles (SAP).

The pump head may be disposable or reusable, i.e. for multi-patient use. For example, the pump head and the separating means may be comprised or integrated in one unit. Thus, in embodiments, in which the separating means comprises a canister or container for collection of pumped liquid, the pump is removed from the drive unit with the container, e.g. for emptying or exchange thereof. As the detachable and disposable pump head is integrated with the separating means to form one part, the pump head and the separating means can be disposed off at the same time, after the system of the invention has been in use. This is advantageous, as it makes the handling of the system simpler. At the same time, it reduces the risk of erroneously reusing the pump head of the means for applying negative pressure on a different patient or user, after the system has been used.

If the pumping element or pump head is reusable, it is preferably capable of being sterilized. For example, the pump head may be capable of being sterilized by radiation. In another embodiment, the pump head can be sterilized by autoclave. In another embodiment, the pump head can be sterilized by steam. In one embodiment, the pump head may be sterilized by means of ethylene oxide (ETO sterilization). In yet another embodiment, the pump head can be sterilized by washing. The pump head as well as the drive unit can be reused. By providing the two parts being attached detachably, it is possible to separate the possibly virus- or bacteria-carrying pump head from the drive unit, which has not been in contact with virus or bacteria. After the pump head has been detached from the drive unit, it may be cleaned properly so as to remove any possible residue of virus or bacteria. In preferred embodiments, the pump head mainly comprises mechanical parts and no delicate electronics. Thus, it can be cleaned effectively on both the inside and the outside without harming the functionality of the pump head, which consequently makes it reusable.

The pump head may be permanently integrated in the container, i.e. non-detachably comprised therein, or it may be detachably integrated in the container.

The drive unit may comprise an electric motor. The electric motor may be connected to a conventional power plug, or it may be connected to a battery pack, or it may be connected to a combination of a power plug and a battery pack. The battery pack will be most advantageous when the system is used for the treatment of a mobile user.

A control unit of the device may e.g. comprise means for controlling the electric motor. For example, operation of the electric motor may be determined by the control unit based on e.g. a pressure determined at the wound, in the separating means, in a conduit connecting the wound cover to the separating means, or in any other suitable location. The motor may be intermittently operable to cause the pump to pump intermittently, or it may operated continuously at variable speed. The control unit is preferably an integrated part of the drive unit.

The container may have at least one inlet for fluid and gas to enter the container, and at least one outlet for gas to exit the container into the pumping element. The inlet and outlet may be arranged such with respect to the container and the pumping element that liquid is separated from gas upstream of the pumping element, and the pump may further comprise a gas outlet allowing gas pumped through the pumping element to escape from the pump.

The container may alternatively have at least one inlet for fluid and gas to enter the container, and at least one outlet for gas and liquid to exit the container into the pumping element. An exit port of the pumping element may be connected to the container, so that liquid pumped through the pumping element is conveyed into the container, and the pump may further comprise a gas outlet allowing gas pumped through the pumping element to escape from the pump.

Structure may be provided for preventing liquid in the container from entering the pumping element at the pressure side of the pumping element. Such structure may include mechanical barrier means, such as one or more siphon traps, valves or other one-way arrangements.

A gas escape outlet for allowing gas to escape to an ambient atmosphere may be provided, the gas escape outlet being provided with a filter.

In the system of the present invention, the wound cover may comprise a semi permeable cover foil. For example, the cover foil may be impermeable to liquid to penetrate from the wound to the exterior of the cover foil, but permeable to vapour.

Screen means, such as a polymer foam, such as an open-cell polymer foam, may be provided within the wound cover. Suitable screen means are disclosed in EP 0 620 720, which is hereby incorporated by reference. Alternatively, other flexible structures allowing transport of exudate may be provided.

As described above, a pressure sensing element may be provided for detecting a pressure level in the system and for communicating said pressure value to the control unit of said device. The pressure sensing element may be arranged to detect a pressure level within the wound cover or within the fluid collecting container of the system. In case the pressure sensing element is arranged to detect a pressure level within the wound cover, i.e. at the wound, the wound cover may be connected to the collecting chamber via a multi lumen conduit, such as a double-lumen conduit. The multi lumen conduit may comprise a first passage for applying negative pressure within the wound cover, and a second passage for transmitting a negative pressure to the pressure sensing element.

Alternatively, the pressure sensing element may be comprised within or at the wound cover, in which case the pressure sensing element may be adapted to transmit an electronic signal to the control unit of said device, e.g. through a wire or a wire-less connection.

An irrigation system for irrigating the wound may be applied. One suitable irrigation system is disclosed in WO 03/057070, which is hereby incorporated by reference.

Embodiments of the system according to the present invention may comprise a first, a second, and a third compartment, the pump comprising a motor-driven pumping element and a motor for providing a driving force to the pumping element, the motor being comprised in the first compartment, and the pumping element being comprised in the second compartment. The suction system may further comprise a container for collecting liquid pumped from the treatment site, the container being comprised or integrated in the third compartment, the second compartment being detachably connected to the first and/or third compartment, and the third compartment being detachably connected to the first and/or the second compartment.

It will hence be appreciated that the container for collecting liquid as well as the pumping element may be detachably mounted for replacement or cleaning thereof. Thanks to the provision of three compartments as recited above, the container may be exchanged or emptied, without removing the pumping element or without replacing the pumping element. As the pumping element constitutes a relatively expensive component compared to the container, the present invention offers an inexpensive pump and system in the sense that no replacement of the pumping element is required if the container is to be replaced.

It will be understood that, in embodiments of the present invention, the first compartment constitutes a drive unit, i.e. a unit housing those elements and components which are required in order to cause the pumping element to pump. As described in further detail below, the pumping element may connect into a driving connection with the driving components when the second compartment is attached to the first and/or to the third compartment.

In the present context, the first, second and third compartments should be understood to constitute or include parts of the housing of the pump. Hence, each compartment preferably has at least one wall portion, which, when the compartments are assembled to constitute the housing of the pump, constitutes an outer wall of the housing of the pump. Within each compartment, one or several sub-compartments may be provided, such as containers, pumping cavities, motor casing etc.

The releasable attachment of the compartments to each other may be achieved in a number of different ways. Mechanical engagement means may be provided, including e.g. a latch and/or a lock structure for releasably securing the compartments in relation to one another. In addition, or as an alternative, magnetic means may be provided.

The pumping element may comprise a peristaltic element, which applies negative pressure by successive contraction and expansion of a flow cross-section of a tube, contraction and expansion being e.g. achieved by a rotatable element with one or more protruding portions acting on an outer surface of the tube to thereby cause the flow cross-section in the tube to successively contract and expand. Alternatively, the pump may include at least one reciprocatable element, such as a piston or diaphragm, arranged to cause successive contraction and expansion of a pumping cavity, whereby respective one-way valves are provided at the inlet and the outlet of the pumping cavity.

Alternatively, the pumping element may e.g. Include a piston or diaphragm, whereby the first compartment may include those elements required to cause the pumping element to reciprocate or cause a pumping action. Preferably, a liquid and/or gas tight seal is provided between the pumping element and the drive unit, so that gas and/or liquid is prevented from entering the drive unit. For example, the pump head, including e.g. a piston or a membrane, may seal circumferentially against a wall partition. Contamination of the drive unit may thereby be avoided, or the risk of contamination of the drive unit may be reduced. Hence, as the third compartment with the container is typically replaced when the pump is to be used with another patient, only the pump head of the second compartment needs sterilization between uses of the device with different patients. Alternatively, the second compartment may be disposable, and hence it may be exchanged when the pump is moved from one patient to another.

The first, second and third compartments may be arranged in various configurations. For example, the compartments may have a generally rectangular cross section, with a first surface of the second compartment lying flush with a surface of the first and/or third compartment. A second surface of the second compartment, which is opposite to the first surface may lie flush with a surface of the other one of the first and third compartment. Alternatively, the compartments may be generally pie or arc shaped. For example, each compartment may form a pie or arc element spanning an angle of about 120°, so that each compartment contacts two of the other compartments. Conveniently, the second compartment is directly attached to the first compartment, so that attachment of the second compartment to the first compartment brings the pumping element into a driving cooperation with the motor drive of the first compartment.

As a safety measure, the compartments may be configured such that the second compartment is only detachable from the first compartment when the third compartment is detached form the first/and second compartment. Hence, it may be avoided that the pumping element is detached, while the apparatus is pumping liquid and other matter from the treatment site. In one embodiment, a latch for releasing the second compartment from the first compartment is covered by the third compartment, when the third compartment is attached to the second compartment. In another embodiment, electronic control means, including e.g. one or more switches and/or light diodes, are arranged to detect if the third compartment is in place, and, if so, to prevent removal of the second compartment.

The pump may be configured to pump only gas through the pumping element and/or to pump gas and liquid therethrough. In the first alternative, the container has at least one inlet for liquid and gas to enter the container, and at least one outlet for gas to exit the container into the pumping element, with the inlet and outlet being arranged such with respect to the container and the pumping element that liquid is separated from gas upstream of the pumping element. In such an embodiment, the pump further comprises a gas outlet allowing gas pumped through the pumping element to escape from the pump. In this embodiment, it may be at least substantially prevented that the fluid and possibly also solid matter pumped from the wound enters the pump, whereby wear on the pump and the risk of occlusion of the pump may be avoided.

In the other alternative, the pumping element has at least one inlet for liquid and gas to enter the pumping element, and at least one outlet for liquid and gas to exit the pumping element, whereby an exit port of the pumping element is connected to the container, so that liquid pumped through the pumping element is conveyed into the container. In such an embodiment, the pump further comprises a gas outlet allowing gas pumped through the pumping element to escape from the pump. This embodiment may be suitable for applications, in which occlusion of elements of the pump is not a risk.

As it will be understood from the above description, the pumping element may preferably be integrated in the second compartment. Preferably, also a pumping cavity, which is caused to contract and expand by reciprocation of the pumping element, is entirely integrated in the second compartment. It may thereby be achieved that pumped matter is prevented from entering and contaminating the first compartment, which houses the drive components of the pump.

The gas outlet may be provided in any of the compartments, however to avoid contamination of the first compartment housing the drive components of the pump, it is preferable to have the gas outlet in the second or the third compartment. One or more filters may be provided as described below.

In embodiments, in which the pumping element comprises a pump head, through which gas and/or liquid from the treatment site is pumped during operation of the pump, a liquid and/or gas tight seal is preferably provided between the pump head and the first compartment, so that gas and/or liquid is prevented from entering the first compartment. The seal may e.g. be provided by the pumping element, constituted e.g. by a membrane or a piston, sealing against the second compartment, so that the pumping cavity of the second compartment, when attached to the first and/or third compartment in the operative configuration, is sealed to prevent pumped matter from escaping from the second to the first compartment. Alternatively, the pumping element may comprise a peristaltic element, which applies negative pressure by successive contraction and expansion of a flow cross-section of a tube contained in the second compartment. Contraction and expansion of the tube may e.g. be achieved by a rotatable element with one or more protruding portions acting on an outer surface of the tube to thereby cause the flow cross-section in the tube to successively contract and expand, such rotatable element being comprised in the first compartment, or at least driven by drive components in the first compartment.

A pressure sensing structure for detecting a negative pressure within the wound cover may be provided, including e.g. a deflectable member as described in detail above. Alternatively, or additionally, a pressure sensing element for detecting a negative pressure in the container may be provided, also with the option of including a deflectable member as described above. Such pressure sensing element may be connected to a control unit controlling operation of the pump motor to ensure that a desired negative pressure is maintained at the treatment site. In one embodiment, the pressure sensing element may be provided in the first compartment. Hence, the second and third compartments, which in most embodiments are disposable elements, are not rendered unnecessarily expensive by the presence of the pressure sensing element.

In case the pressure sensing element is provided in the first compartment, the pressure sensing element may be arranged at or near an end of a pressure conduit extending at least partly through the second compartment, so that no separate external conduit or tube is required to enter the first compartment. This may reduce the number of parts to be assembled, facilitate operation of the pump, and reduced the risk of contamination of the first compartment.

The pressure conduit may open into the container or into a pressure input port of the second or third compartment. In the latter alternative, the pressure input port may be connected to the treatment site, e.g. a wound covered by a wound cover, in which case the pressure sensing element detects pressure at the treatment site. Likewise, pressure may be detected in other parts of the system. In the first alternative, the pressure sensing element detects pressure in the container, which may be equal to or at least representative of the pressure at the treatment site.

In other embodiments, the pressure sensing element is provided in the second compartment.

### DESCRIPTION OF THE DRAWINGS

Embodiments of the invention will now be described with reference to the drawings, in which:

FIG. 1 schematically illustrates a system according to the invention;

FIG. 2 schematically illustrates a pressure sensor and a deflectable member;

FIGS. 3-14 schematically illustrate various embodiments of deflectable members and their positions relative to sensing devises.

FIGS. 15-22 are cross-sections of embodiments of pumps for use in the system according to the invention;

FIGS. 23 and 24 are schematic views of a further embodiment of a system according to the invention;

FIG. 25 shows a perspective view from the side of one embodiment of separating means for separating gas and liquid upstream of the pump;

FIG. 26 shows a perspective bottom view of the separating means in FIG. 25;

FIG. 27 shows a perspective side view of the drive unit, the pump head and the container according to the embodiment of the separating means in FIG. 25;

FIG. 28 shows another embodiment of the separating means and conduit, where the separating means and conduit are combined in one unit.

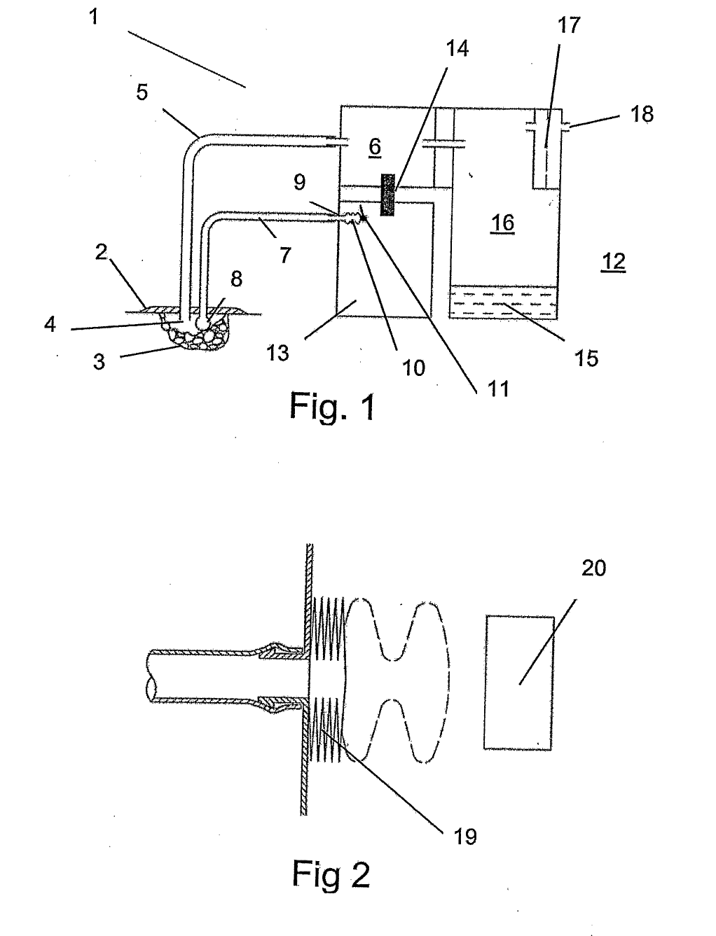

FIG. 1 illustrates a suction system 1 comprising a wound cover 2 which is attached to the circumference of a wound 3 and thus forms an enclosure 4. The drainage conduit 5 connects the enclosure 4 to the pumping structure, in the following referred to as a pump head 6, and the pressure signal conduit 7 is in fluid communication with the enclosure 4 via the inlet 8. The axially opposite end 9 of the pressure signal conduit 7 is sealed with a deflectable member 10 which prevents exudate in the enclosure to escape and thus protects the sensor 11 against contamination. In FIG. 1, the deflectable member and sensor is illustrated schematically only. A pressure difference between pressure in the enclosure 4 and pressure in the ambient space 12 causes deflection of the deflectable member 10, and the sensor 11 is adapted to determine such deflection and thereby determine the pressure difference. The sensor 11 is located in a motor housing, in the following referred to as a drive unit 13 which also contains power driven means for actuating the pump head 6 via the drive structure 14. The drive unit further comprises a battery for portable, self supplied use. The deflectable member 10 is detachably connectable to the drive unit 13 and thereby to the sensor 11. As illustrated, the pressure signal conduit 7 has a smaller cross-sectional size than the drainage conduit 5.

The drained liquids and other exudate 15 are drained from the pump head 6 into a reservoir 16, and the sucked gas is exhausted through the filter 17 and the outlet 18 to the ambient space 12. The filter prevents bacterial contamination of the ambience as well as it may prevent malodour.

FIG. 2 shows details of the deflectable member 19 and sensor 20 in a situation wherein the pressure signal conduit 7 is attached to the sensor. The deflectable member 19 has the shape of a bellow which changes shape based on a pressure difference between the negative pressure in the enclosure 4 and the pressure of the ambient space, i.e. in this case atmospheric pressure. As the negative pressure decreases, the bellow shaped part shortens, and the reduced length is sensed by the sensor 20. The dotted line indicates the bellow shaped part in an extended state and the full-drawn line indicates the compressed state.

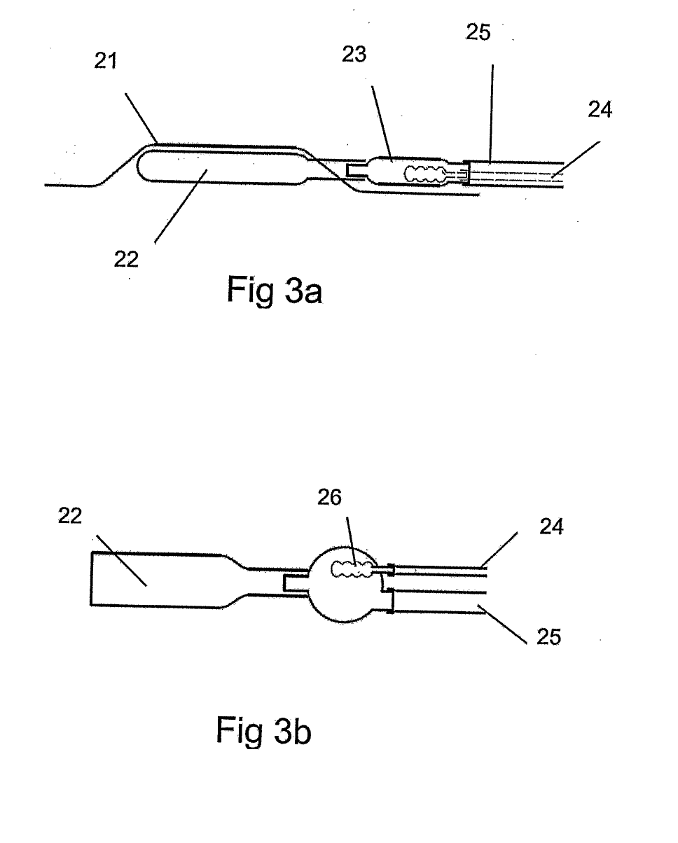

FIG. 3a shows a side view of a deflectable member located at the enclosure. Numeral 21 designates a wound cover, numeral 22 a suction head located in the enclosure, numeral 23 a measuring capsule, numeral 24 a measuring tube, and numeral 25 a suction tube. FIG. 3b shows the same embodiment from the top view of the measuring capsule in which a bellow 26 deflects based on the pressure difference.

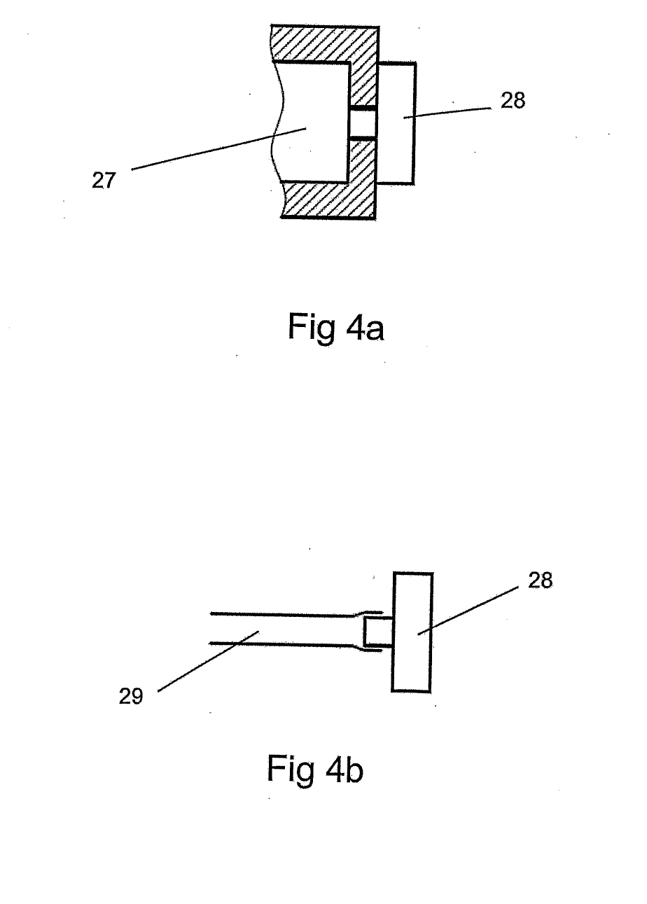

FIG. 4a shows a remote sensor connection. Numeral 27 designates a measuring chamber, and numeral 28 a sensor. FIG. 4b shows an alternative embodiment with a tube connection 29, which leads to a measuring point. It will be appreciated that FIGS. 4a and 4b do not illustrate the deflectable member, but merely illustrate a sensor arrangement.

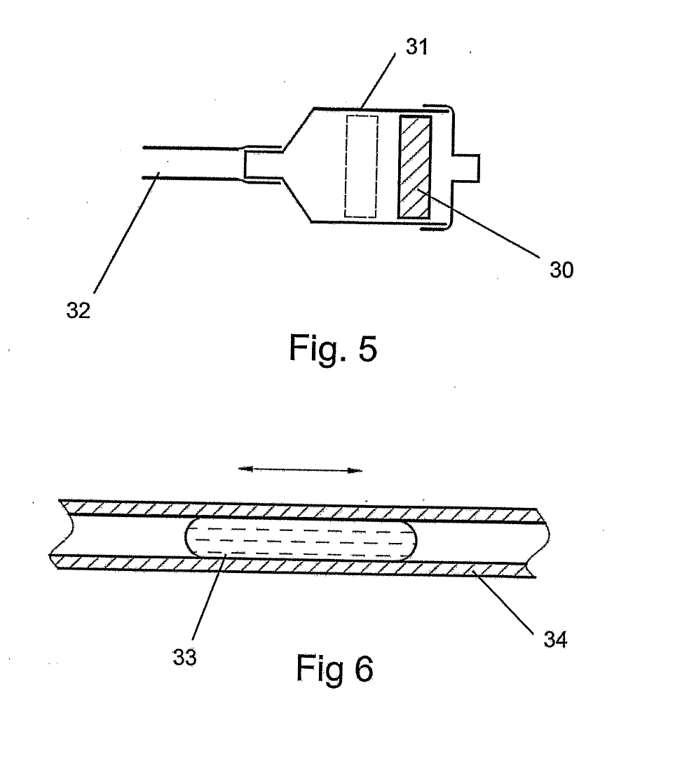

FIG. 5 shows a deflectable member in the shape of a moving piston 30 in a cylinder 31 which is connected to a measuring tube 32.

FIG. 6 shows a liquid piston 33 which moves in a measuring tube 34 based on the pressure difference.

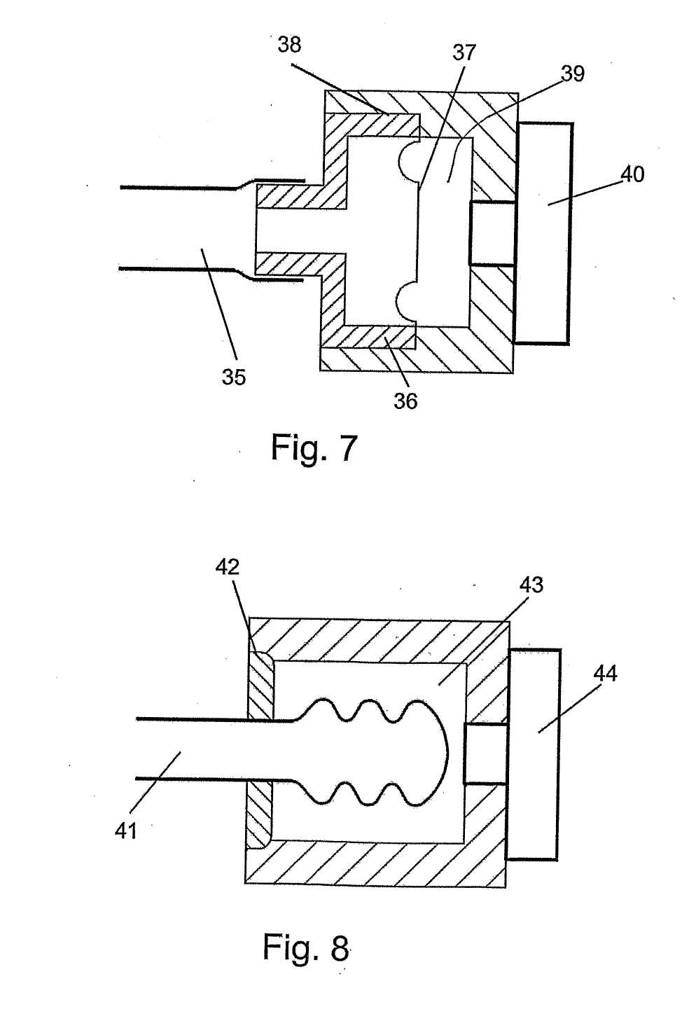

FIG. 7 shows a deflectable member in the shape of a diaphragm located at a remote sensor. Numeral 35 designates a measuring tube, numeral 36 a housing with a diaphragm 37, numeral 38 a snap fit, numeral 39 a measuring chamber and numeral 40 a sensor.

FIG. 8 shows a deflectable member in the shape of a bellow located in front of the sensor. Numeral 41 designates a measuring tube, numeral 42 a snap fit, numeral 43 a measuring chamber, and numeral 44 a sensor.

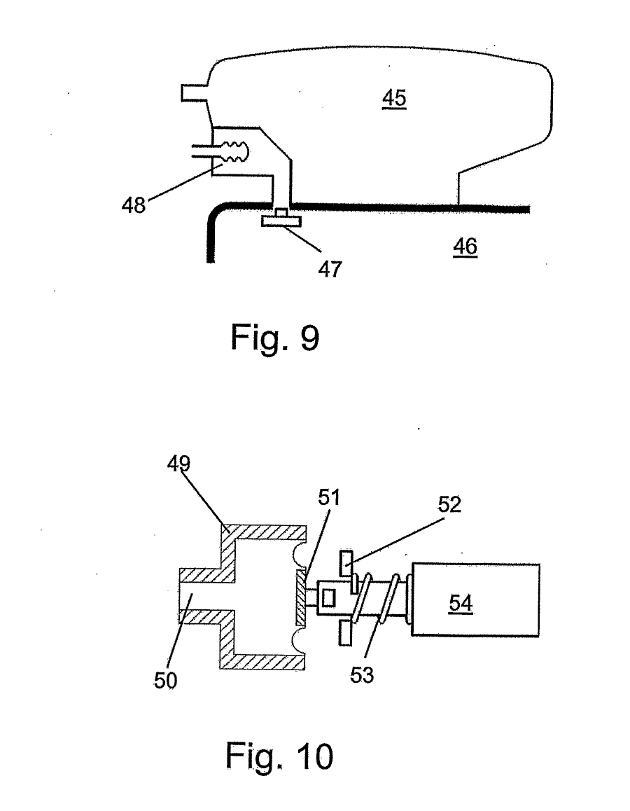

FIG. 9 shows a deflectable member in a disposable pump head or waste container. Numeral 45 designates the pump head or waste container, numeral 46 a drive unit, numeral 47 a sensor, and numeral 48 a separate measuring chamber.

FIG. 10 shows contact measurement with a housing 49 comprising a passage 50 connecting the housing to the enclosure, a diaphragm 51, a spring retainer 52 and a spring 53, and a potentiometer 54. The potentiometer is engaged to and thus moved by the diaphragm by direct contact therewith.

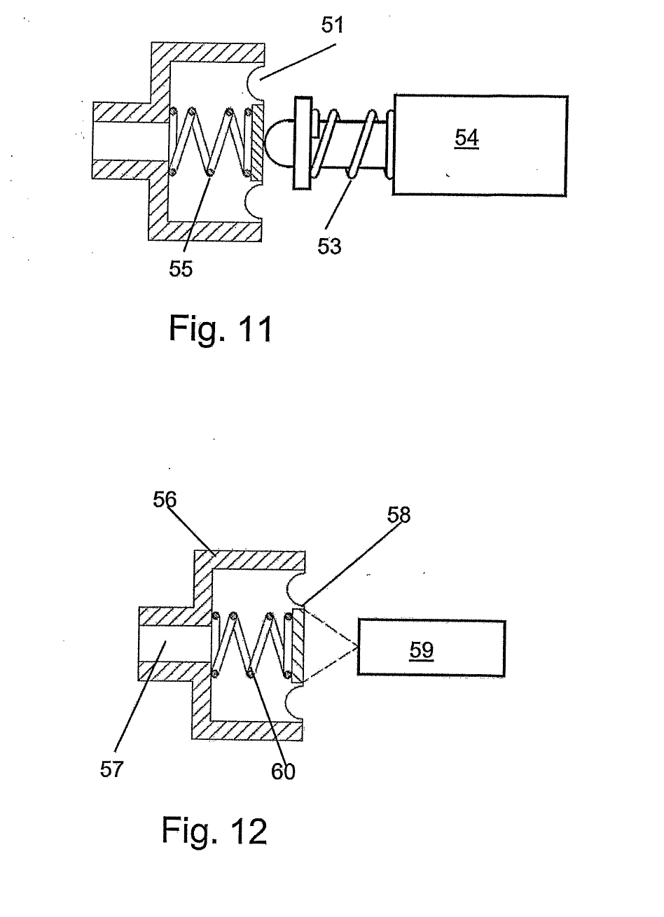

FIG. 11 shows an alternative way of making contact measurement. In this embodiment, an additional spring 55 inside the disposable part provides the sensitivity of the device whereas an additional weaker spring 53 provides contact of the potentiometer against the deflective member to make the potentiometer follow the movement without the need of engagement between the two.

FIG. 12 shows contactless measurement with a distance sensor. Numeral 56 designates a housing, numeral 57 a passage connecting the housing to the enclosure, numeral 58 a diaphragm, numeral 59 a sensor, and numeral 60 a spring providing the sensitivity of the device.

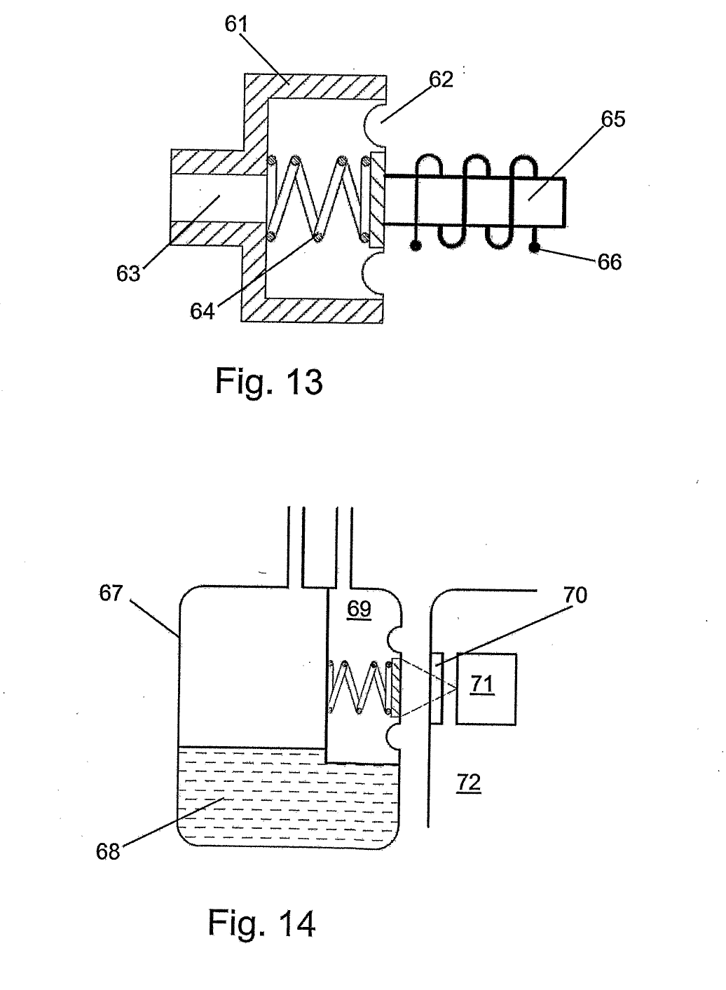

FIG. 13 shows contactless measurement with magnetic reluctance. Numeral 61 designates a housing with a diaphragm 62 and a passage 63 connecting the housing to the enclosure, numeral 64 is a spring, numeral 65 an iron core and numeral 66 a coil.

FIG. 14 shows a contactless measurement with a deflectable member located in a disposable canister or collector for the exudate. Numeral 67 designates the canister with exudate 68, numeral 69 a separate measuring chamber, numeral 70 a window, numeral 71 a sensor, and numeral 72 a drive unit.

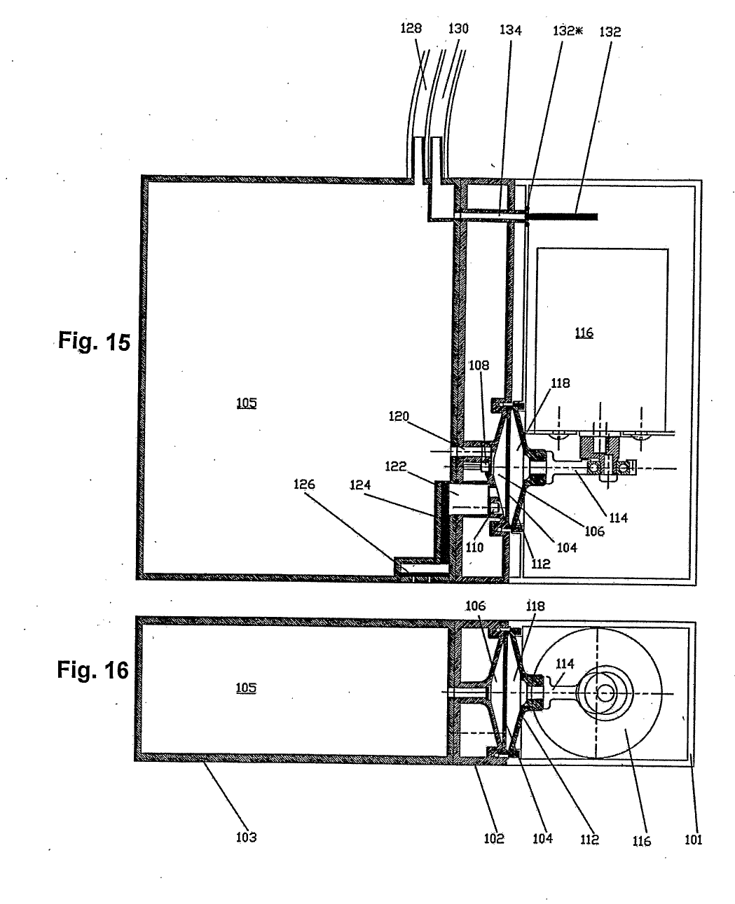

FIGS. 15 and 16 are cross-sectional views in two perpendicular planes of a pump for use in a system according to the invention. The pump comprises three compartments, a first compartment 101, a second compartment 102, and a third compartment 103. As described in further detail below, the first compartment 101 houses drive components to cause a pumping element of the second compartment 102 to pump exudate from a wound. The third compartment 103 comprises a collecting container 105 for collecting liquid pumped from the wound.

The third compartment 103 is detachably attached to the second compartment 102, which in turn is detachably attached to the first compartment 101. The detachable securing of the compartments relative to each other may e.g. be achieved by one or more latches, such as spring-biased latches, magnetic means, one or more locks or any combination of the aforementioned means. In order to prevent the second compartment 102 from being unintentionally removed from the first compartment 101, a release switch or button for releasing the second compartment from its detachable coupling with the first compartment 101 may be provided at that surface of the compartment 102, which abuts and is rendered inaccessible by the third compartment 103, when the third compartment 103 is attached to the second compartment 102.

An electronic control unit may be provided to ensure that the three compartments are not separated, while the pump is operating. For example, release of the third compartment 103 from the second compartment 102 and/or from the first compartment 101 may be rendered impossible by an electronically operated magnet or lock, if the negative pressure in the container 105 and/or at the wound as measured by a pressure sensing element 132 is above a certain threshold value. Alternatively, the compartments may be interlocked by the control unit, if a motor 116 for driving the pump is operating, or if the pump is powered on.

The second compartment 102 includes a reciprocatable disposable diaphragm 104 forming a wall partition of an outer surface of the second compartment. The diaphragm 104 seals circumferentially in a liquid and gas tight manner against the outer surface of the second compartment 102. Upon reciprocation of the diaphragm 104, a pumping cavity 106 is caused to expand and contract to thereby provide a pumping action. At or in the inlets and outlets of the pumping cavity 106 there are provided respective first and second one-way valves 108, 110. When the pumping cavity 106 expands, the second valve 110 remains closed, whereas the first valve 108 opens. Upon contraction of the pumping cavity 106, the first valve 108 closes, and the second valve 110 opens.

The first compartment 101 houses a permanent diaphragm 112 connected to a driving rod 114 eccentrically mounted with respect to a motor 116. The diaphragm 112 attaches circumferentially to an outer surface portion of the first compartment 101. In a preferred embodiment, the diaphragm 112 also seals to the outer surface portion of the first compartment 101, whereby cleaning of the exterior surface portions of the first compartment is facilitated. The disposable diaphragm 104 of the second compartment 102 additionally forms a circumferential seal against an outer surface portion of the permanent diaphragm 112, so that an intermediate cavity 118 may be enclosed between the two diaphragms. It should, however, be understood that during operation of the pump, the diaphragms 104 and 112 normally lie flat against each other with essentially no gap between them. Rotary motion caused by the motor causes the driving rod 114 and hence the permanent diaphragm 112 to reciprocate. Reciprocation of the permanent diaphragm 112 causes the disposable diaphragm 104 to reciprocate and hence the pumping cavity 106 to expand and contract.

An inlet port 120 for the pump extends through the second compartment 102 and opens into the collecting container 105 of the third compartment 103. An outlet port 122 for discharge of gas is provided downstream of the pump. The outlet port 122 also extends through the second compartment 102 and opens into the third compartment 103. In that portion of the outlet port 122, which is in the third compartment 103, there are provided an odour filter 124 and a bacteria filter 126.

It is generally advantageous that any filter, whether upstream or downstream of the pumping cavity 106, are provided in the third compartment. Hence, new filters are provided when the third compartment is exchanged, whereby the durability of the second compartment 102 is extended.

The pumping action created by reciprocation of the diaphragm 104 provides a negative pressure in the collecting container 105 of the third compartment 103. The collecting container 105 is connected to the wound (not shown) via a drainage conduit 128, whereby a negative pressure is generated at the wound site. A pressure conduit 130 is provided to connect the wound site with a pressure sensing element 132 via a pressure port extending through the second and third compartments 102, 103. A deflectable member 132* forming an essentially air tight barrier upstream of the sensor 132, i.e. between the sensor 132 and the wound enclosure (not shown) is preferably provided. The deflectable member and pressure sensor 132 may be embodied as described above with reference to FIGS. 1-14.

The conduits 128 and 130 may conveniently be constituted by a multi lumen tube, such as a double-lumen tube. However, It is also envisaged that two separate tubes may be provided for the two conduits, in which the pressure conduit may extend directly into the second compartment 102 or even directly into the first compartment 101. However, in order to reduce the risk of contamination of the components housed in the first compartment 101, including the pressure sensor 132, the pressure preferably connects to the second or the third compartment.

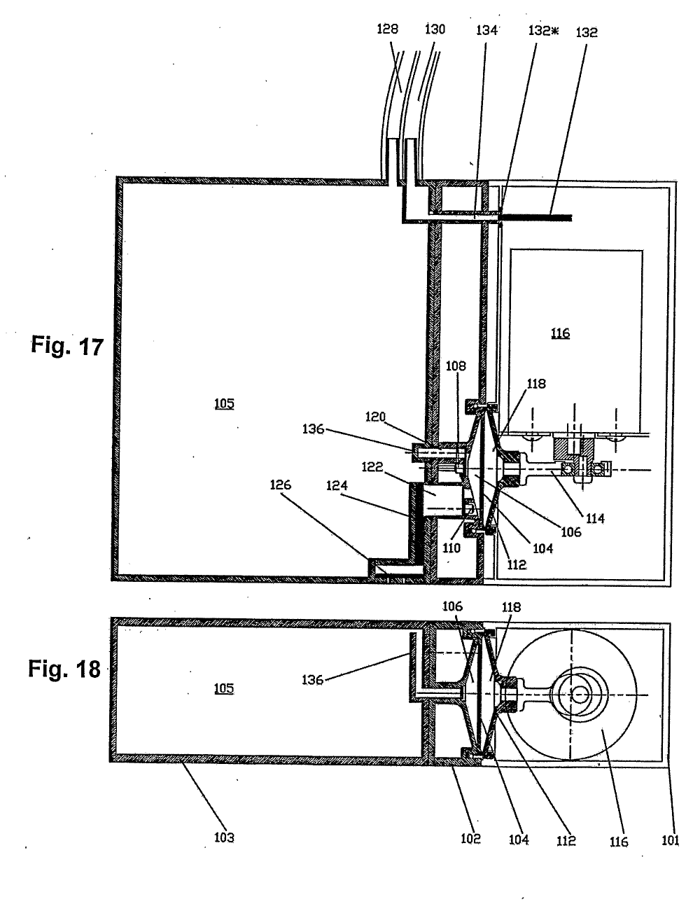

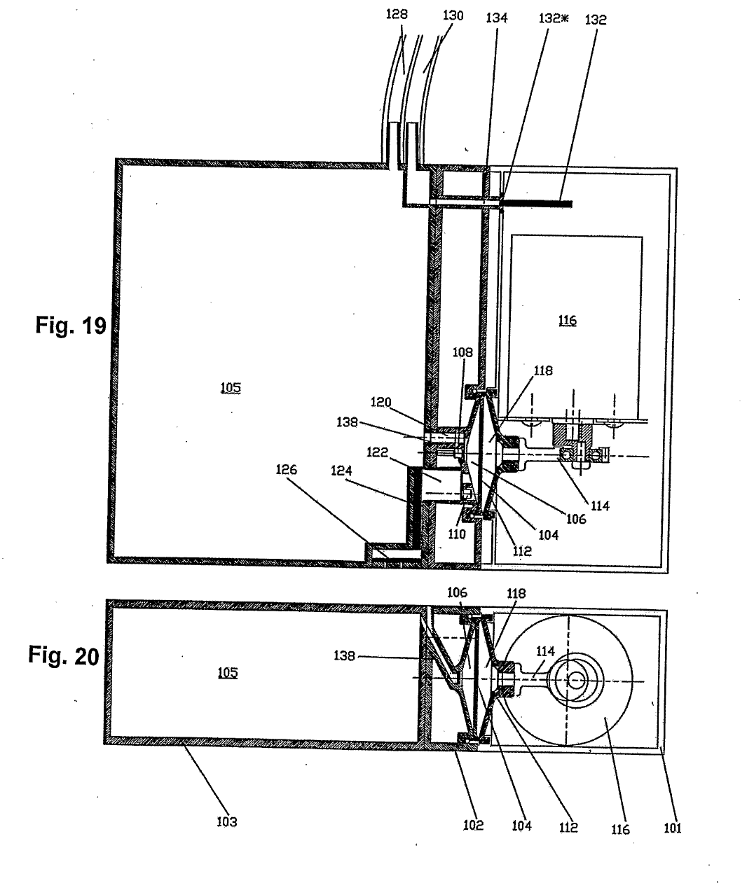

FIGS. 17-20 show a modified embodiment of the pump of FIGS. 15 and 16, in which walls 136 (FIGS. 17 and 18) and 138 (FIGS. 19 and 20) are provided to ensure that gas is sucked from a top portion of the container 105.

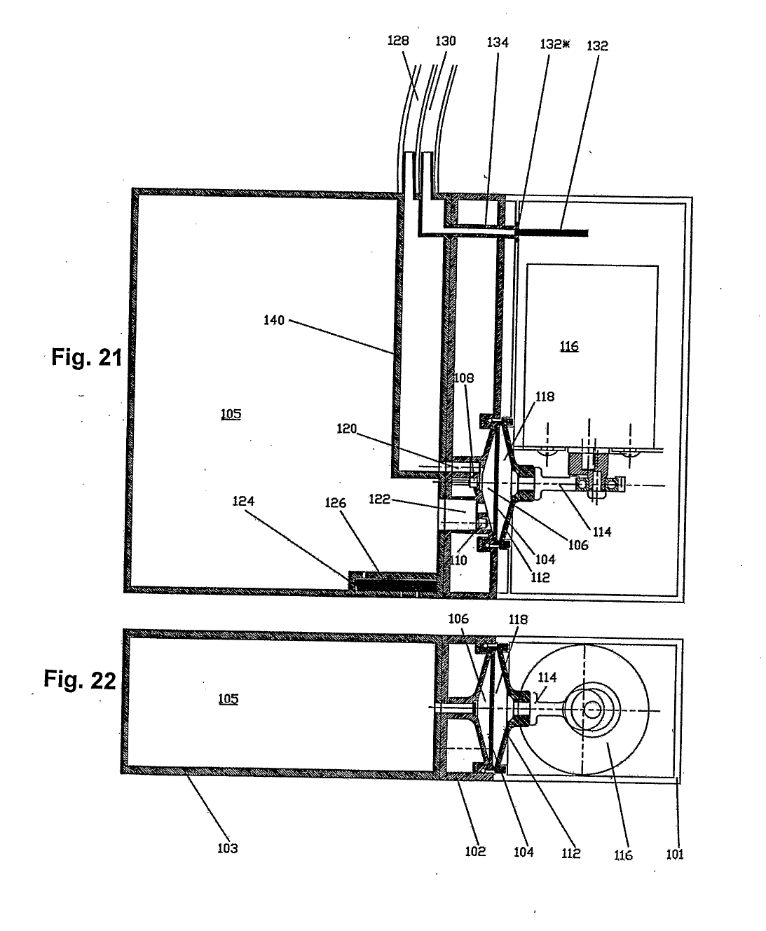

In the embodiment of FIGS. 21 and 22, a wall 140 is provided in the third compartment to form a passage connecting the drainage conduit 128 to the pump inlet 120. Thereby any matter pumped from the wound, including liquid is pumped through the pumping cavity 106. Hence, the outlet port 122 of the pump is arranged to discharge liquid and gas into the collecting container 105 of the third compartment 103. In this embodiment, a gas discharge port, comprising the odour filter 124 and bacterial filter 126, connects the collecting container 105 with the exterior to allow discharge of gas from the container.

It will be appreciated that the pressure sensing structure described above with reference to FIGS. 1-14 may be applied in the pump structure described with reference to FIGS. 15-22.

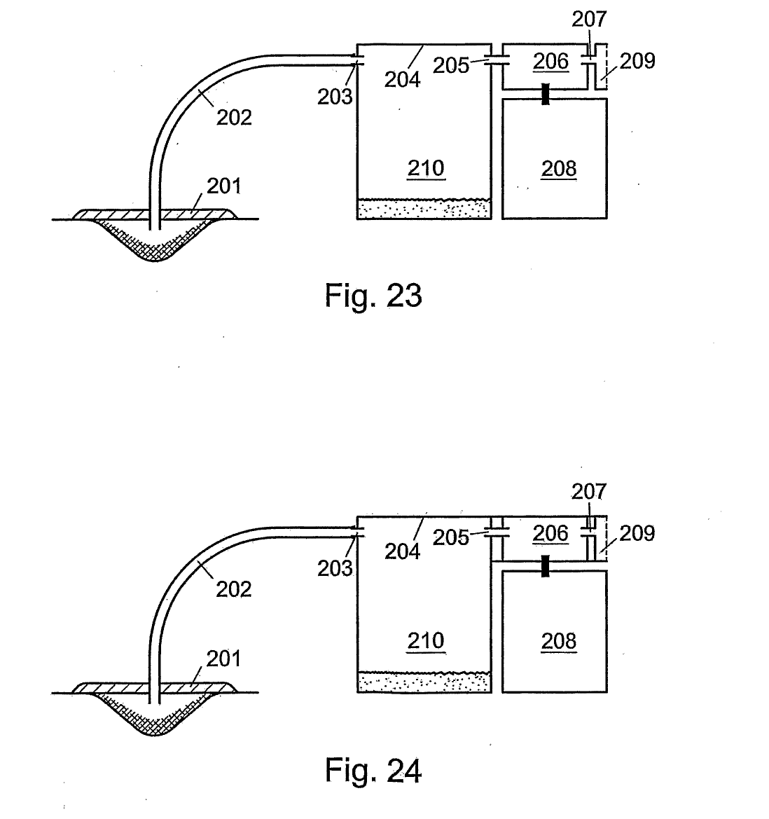

FIG. 23 shows a first embodiment of the invention, where the wound cover 201 is connected by means of the conduit 202 via the fluid inlet 203 to the separating means 204, which comprises a container 210. The separating means separates the liquid from the gas, and thus substantially only gas passes through the gas outlet 205 to the pump head 206. In this embodiment, the separated liquid and possible solid material is collected in the container 210. The drive unit 208 drives the pump head 206 so that the system provides negative pressure. The pump head 206 also comprises an outlet 207 and a filter 209.

FIG. 24 shows a second embodiment of a system according to the invention, where the pump head 206 is integrated with the separating means 204 to form one unit. For example, the pump head 206 may be integrated in the container 210.

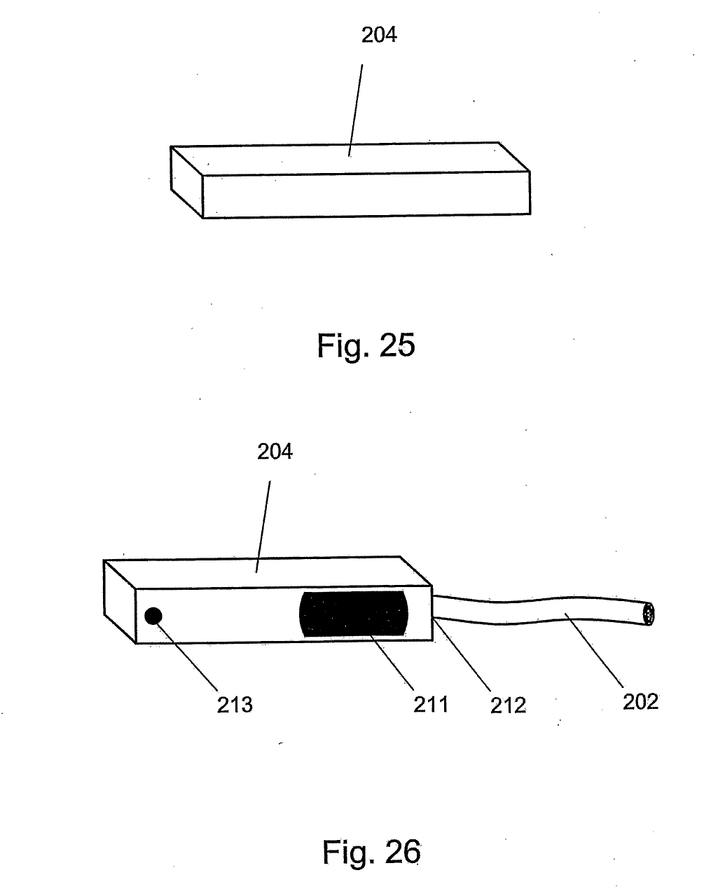

FIG. 25 shows a perspective view from the side of one embodiment of the separating means 204.

FIG. 26 shows a perspective bottom view of the separating means 204 in FIG. 24. In this embodiment, the separating means 204 comprises an elongate part with a second fluid inlet 212, a fluid outlet 211, and a second gas outlet 213. The fluid outlet 211 is placed between the second fluid inlet 212 and the second gas outlet 213. When fluids from the wound pass through the second fluid inlet 212, they are subsequently separated into liquids, which pass through the fluid outlet 211 into a container (not shown), and gases, which pass trough the second gas outlet 213. The separation can be achieved by means of gravity so that liquids fall down through the fluid outlet 211, while gases continue to the second gas outlet 213.

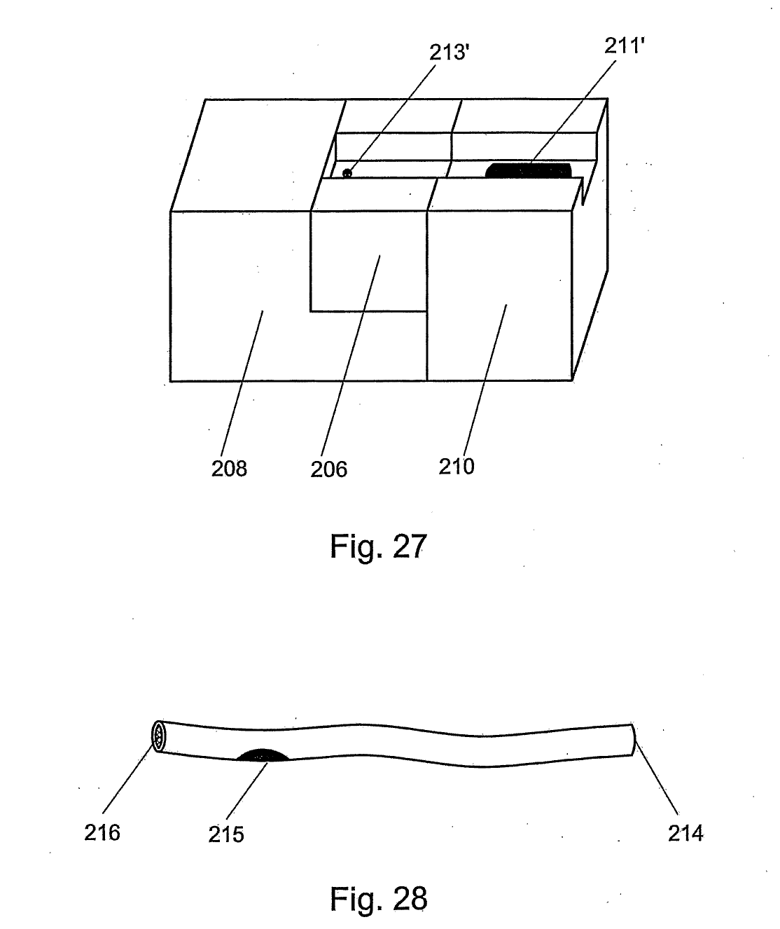

FIG. 27 shows a perspective side view of the drive unit 208, the pump head 206 and the container 210 according to the embodiment accommodated for the separating means 204 in FIG. 25. The recess in the center of the top face of the drive unit 208, the pump head 206 and the container 210 are provided to accommodate the separating means 204 of FIG. 26. The pump head 206 is provided with a pump gas inlet 213′ provided and placed so as to correspond to the gas outlet 213 of the separating means. The container 210 is provided with a fluid inlet 211′ which corresponds to the fluid outlet 211 of the separating means 204.

FIG. 28 shows another embodiment of the separating means 204 and conduit 202, where the separating means 204 and conduit 202 are combined in one unit. In this embodiment the separating means 204 and the conduit 202 are provided as a tube, which in a distal section comprises a second fluid outlet 215 and a third gas outlet 216. The separation of fluids into liquids, and gases is done in a similar way to that of the embodiment shown in FIGS. 25-27. In another embodiment a filter is placed before the fluid inlet 211′ in order to assure that no liquid is entered into the pump head 206.

It will be appreciated that the pump structure of FIGS. 15-22 may be applied in the system described with reference to FIGS. 23-28.

1. A suction system for removal of exudate from a wound, the system comprising:
a wound cover which is attachable to a wound circumference of a living being thereby to form an enclosure,
a pump in fluid communication with the enclosure to provide a pressure difference between a negative pressure in the enclosure and an air pressure of an ambient space,
a deflectable member arranged such with respect to the enclosure that the pressure difference may cause deflection of the deflectable member, and
a pressure sensing device located outside the enclosure and arranged to provide a signal in response to the pressure or the deflection of the deflectable member,
wherein the deflectable member forms an essentially air tight barrier between the sensor and the enclosure.

2. A suction system according to claim 1, wherein the deflectable member is detachably engageable to the sensing device.

3. A suction system according to claim 1, wherein the deflectable member is detachably mountable in a fixed position relative to the sensing device.

4. A suction system according to claim 1, wherein the deflectable member forms part of a wall, which separates the enclosure from the ambient space.

5. A suction system according to claim 1, wherein the deflectable member forms a wall-part of a pressure signal conduit, which is in fluid communication with the enclosure.

6. A suction system according to claim 5, wherein the pressure signal conduit forms an inlet into the enclosure, which inlet comprises a separation structure which prevents entrance of liquid substances into the pressure signal conduit.

7. A suction system according to claim 5, comprising a drainage conduit providing the fluid communication between the enclosure and the pump, the drainage conduit and the pressure signal conduit being formed in one single elongated member.

8. A suction system according to claim 7, wherein the pressure signal conduit has a smaller cross-sectional area than the drainage conduit.

9. A suction system according to claim 1, wherein the pump is contained in a pump housing, the pump housing further containing the sensor and a coupling structure for fixing the deflectable member relative to the sensor in an operative position.

10. A suction system according to claim 9, wherein the coupling structure is further adapted to connect the drainage conduit to the pump.

11. A suction system according to claim 10, wherein the coupling structure is adapted to disconnect both the drainage conduit and the pressure signal conduit from the pump housing by a single user interaction.

12. A suction system according to claim 9, wherein the pump housing is divided into a pump head comprising a pumping structure which is connectable to the drainage conduit and a drive unit which contains power driven means for actuating the pumping structure and which is connectable to the pressure signal conduit, the pump head being detachably connectable to the drive unit.

13. A suction system according to claim 1, comprising a control structure for controlling the pump based on the pressure signal to provide a desired negative pressure.

14. A suction system according to claim 1, wherein the deflectable member is forced in a first direction by a flexible spring means.

15. A suction system according to claim 14, wherein the deflectable member is forced in a direction which is opposite the first direction by the difference between a negative pressure which is lower than the pressure of the ambient space.

16. A suction system according to claim 1, wherein the pump comprises a pump head, the system further comprising a separating means comprising a fluid inlet and a gas outlet, the pump head being connected to the gas outlet of the separating means, and wherein the pump head is detachably attached to the drive unit, so that in use the fluid inlet of the separating means is connected to a wound cover, e.g. via a conduit.

17. A suction system according to claim 1, wherein the pump includes a diaphragm pump.

18. A suction system according to claim 1, comprising a drive unit with an electric motor.

19. A suction system according to claim 16, wherein the pump head is disposable.

20. A suction system according to claim 16, wherein the separating means and the pump head is comprised in one unit.

21. A suction system according to claim 16, wherein the system comprises a filter.

22. A suction system according to claim 21, wherein the filter is placed before the pump head.

23. A suction system according to claim 21, wherein the filter is placed at an outlet of the pump head.

24. A suction system according to claim 16, wherein the drive unit is provided with a control unit.

25. A suction system according to claim 24, wherein the control unit is an integrated part of the drive unit.

26. A suction system according to claim 16, wherein the pump head is reusable.

27. A suction system according to claim 16, wherein the pump head can be sterilized.

28. A suction system according to claim 16, wherein the separating means comprises a container.

29. A suction system according to claim 28, wherein the container has:
at least one inlet for liquid and gas to enter the container; and
at least one outlet for gas to exit the container into the pump head;
said inlet and outlet being arranged such with respect to the container and the means for applying negative pressure that liquid is separated from gas upstream of the pumping element;
the means for applying negative pressure further comprising a gas outlet allowing gas pumped through the pumping element to escape from the pump.

30. A suction system according to claim 28, wherein the container has:
at least one inlet for liquid and gas to enter the container; and
at least one outlet for gas and liquid to exit the container into the pumping element;
whereby an exit port of the pumping element is connected to the container, so that liquid pumped through the pumping element is conveyed into the container;
the pump further comprising a gas outlet allowing gas pumped through the pumping element to escape from the pump.

31. A suction system according to claim 28, wherein the container comprises an absorbent element.

32. A suction system according to claim 28, further comprising structure for preventing liquid in the container from entering the pumping element.

33. A suction system according to claim 1, further comprising a gas escape outlet for allowing gas to escape to an ambient atmosphere, the gas escape outlet being provided with a gas filter.

34. A suction system according to claim 1, wherein the pumping element includes a piston.

35. A suction system according to claim 1, wherein the pumping element includes a diaphragm.

36. A suction system according to claim 1, wherein the wound cover comprises a semi permeable cover foil.

37. A system according to claim 1, further comprising a flexible structure within the wound cover allowing transport of exudate.

38. A system according to claim 37, wherein said flexible structure comprises a polymer foam.

39. A system according to claim 38, wherein said polymer foam comprises an open-cell polymer foam.

40. A suction system according to claim 9, wherein the pressure sensing device is comprised in said pump hosing, and wherein the wound cover is connected to said separating means via a multi lumen conduit, said multi lumen conduit comprising a first passage for applying negative pressure within the wound cover, and a second passage for transmitting a negative pressure to the pressure sensing element.

41. A suction system according to claim 1, wherein the pressure sensing element is comprised within or at the wound cover, the pressure sensing element being adapted to transmit an electronic signal to the control unit of said device.

42. A suction system according to claim 1, wherein the pressure sensing element is arranged to detect a pressure level within the wound cover.

43. A suction system according to claim 42, wherein the pressure sensing element is arranged to detect a pressure level in a fluid collecting container of the system.

44. A suction system according to claim 1, further comprising an irrigation system for irrigating the wound.

45. A suction system according to claim 1, comprising a first, a second, and a third compartment, the pump comprising a motor-driven pumping element and a motor for providing a driving force to the pumping element, the motor being comprised in said first compartment, and the pumping element being comprised in the second compartment, the suction system further comprising a container for collecting liquid pumped from the treatment site, the container being comprised or integrated in the third compartment, the second compartment being detachably connected to the first and/or third compartment, and the third compartment being detachably connected to the first and/or the second compartment.

46. A suction system according to claim 45, wherein the second compartment is only detachable from the first compartment when the third compartment is detached form the first/and second compartment.

47. A suction system according to claim 45, wherein the container has:
at least one inlet for liquid and gas to enter the container; and
at least one outlet for gas to exit the container into the pumping element;
said inlet and outlet being arranged such with respect to the container and the pumping element that liquid is separated from gas upstream of the pumping element;
the system further comprising a gas outlet allowing gas pumped through the pumping element to escape from the pump.

48. A suction system according to claim 45, wherein the pumping element has:
at least one inlet for liquid and gas to enter the container; and
at least one outlet for liquid and gas to exit the pumping element;
whereby an exit port of the pumping element is connected to the container, so that liquid pumped through the pumping element is conveyed into the container;
the system further comprising a gas outlet allowing gas pumped through the pumping element to escape from the pump.

49. A suction system according to claim 1, wherein the pumping element is integrated in the second compartment.

50. A suction system according to claim 49, wherein the gas outlet is provided in said second compartment.

51. A suction system according to claim 49, wherein the gas outlet is provided in said third compartment.

52. A suction system according to claim 45, wherein the pumping element comprises a pump head, through which gas and/or liquid from the treatment site is pumped during operation of the system, and wherein a liquid and/or gas tight seal is provided between the pump head and the first compartment, so that gas and/or liquid is prevented from entering the first compartment.

53. A suction system according to claim 45, further comprising a pressure sensing element for detecting a negative pressure within the wound cover.

54. A suction system according to claim 1, further comprising a pressure sensing element for detecting a negative pressure in the container.

55. A suction system according to claim 53, wherein the pressure sensing element is provided in the first compartment.

56. A suction according to claim 55, wherein the pressure sensing element is arranged at or near an end of a pressure conduit extending at least partly through the second compartment.

57. A suction system according to claim 55, wherein said pressure conduit opens into the container.

58. A suction according to claim 56, wherein said pressure conduit opens into a pressure input port of the second or third compartment.

59. A suction system according to claim 53, wherein the pressure sensing element is provided in the second compartment.

60. A suction system according to claim 53, further comprising an essentially gas and/or liquid tight barrier for preventing contamination by gas and/or liquid of the pressure sensing element.

61. A suction system according to claim 1, wherein the second compartment comprises a latch for detaching the second compartment from the first compartment, and wherein the latch is inaccessible when the third compartment is attached to the second compartment.

62. A method of removing exudate from a wound, said method comprising the steps of:
forming an enclosure around a wound by attaching a wound cover to a circumference of the wound,
providing a pressure difference between a negative pressure in the enclosure and an air pressure of an ambient space,
arranging a deflectable member such with respect to the enclosure that the pressure difference causes deflection of the deflectable member, and
providing pressure signal in response to deflection of the deflectable member,
wherein the deflectable member is arranged to form a bacterial barrier between the sensor and the enclosure.

63. A method according to claim 62, wherein the pump is controlled based on the pressure signal to provide a desired negative pressure.

64. A method according to claim 62, wherein the sensing signals are calibrated.
